(19) United States
(12) Patent Application Publication
(10) Pub. No.: US 2003/0232045 A1
(43) Pub. Date: Dec. 18, 2003

(54) Methods and compositions for in vivo clearance of pathogens

(76) Inventors: Elliot R. Ramberg, Hollywood, FL (US); Martin J. Lopez, Sunrise, FL (US)

Correspondence Address: Buchanan Ingersoll, P.C., One Oxford Centre, 20th Floor, 301 Grant Street, Pittsburgh, PA, 15219, US

(21) Appl. No.: 10/367,418

(22) Filed: Feb. 14, 2003

Related U.S. Application Data
(60) Provisional application No. 60/357,336, filed on Feb. 15, 2002.
(60) Provisional application No. 60/358,859, filed on Feb. 22, 2002.

(51) Int. Cl. A61K 39/395; A61K 39/42; A61K 39/40

(52) U.S. Cl. 424/130.1; 424/155.1; 424/159.1; 424/164.1

(57) ABSTRACT

The present invention provides methods and compositions using biological factors, such as complement components, and manipulation of cells of erythroblastic lineage and myeloid lineage to facilitate clearance of pathologic targets from the blood stream of a patient in specific phagocytic compartment.

### BACKGROUND OF THE INVENTION

1. Field of the Invention

The present invention relates to the field of immunology. In particular, it is directed to methods and compositions for the in-vivo clearance of pathologic and other targets from the peripheral blood. These targets may include the following but are not limited to microbial organisms such as virus, bacteria, rickettsia and fungi, agents of biological and chemical warfare, dysplastic and metastatic cancer cells, autoimmune antibodies and any molecule mediating a pathologic or other process, or present in the body. Appropriate targets are those that can be bound by a binding partner to form complexes such as immune complexes (IC) that can then be removed from the circulation through processes such as phagocytosis. In particular, the invention comprises methods and compositions using biological factors, such as antibodies and complement components, and manipulation of cells of erythroblastic lineage and myeloid lineage to facilitate clearance of the pathologic targets from the blood stream in multiple phagocytic compartments.

2. Brief Description of the Background Art

Vertebrates have evolved with a complex defense system for protection from invading microbes. In general, this defense system is comprised of two parts, the humoral immune system, and the cellular immune system. The immune system has evolved with two distinct mechanisms for in vivo clearance of pathologic targets in the circulation. The first mechanism is the direct clearance of the immonogenic target, mediated by the humoral response. Herein the body must possess target specific antibody that complexes with the target forming the immune complex (IC) which after complement opsonization is cleared in phagocytic cellular compartments in the body. These include the circulating polymorphonuclear granulocytes (PMNs) and the hepatic and splenic macrophages. This effect is mediated by Fcγreceptors and C3b (or CR1) receptors on the phagocytic cell surface. Tables VI: A and VI: B represent this direct target clearance. This is a natural clearance mechanism requiring immunity to the target.

The second process for in vivo clearance of pathologic targets represented in Table VI: C, involves indirect clearance of the complement opsonized IC by attachment to the primate erythrocyte (E) CR1 surface receptors (E CR1). The reaction is rapid and the IC/C3b complex attached to E CR1 is rapidly shunted to the liver and spleen for phagocytosis via the erythrocyte-immune-complex (E-IC) clearance reaction by the fixed tissue monocytes.

Based on the indirect in vivo clearance mechanism Ronald Taylor presented a strategy wherein a heteropolymer (HP), always defined as IgG anti target-IgG anti CR1, is attached to the primate E forming E HP. The sensitized E HP will rapidly bind the specific target in the circulatory system at the “privileged” CR1 site. Once bound to the erythrocyte, the CR1-HP-target immune complexes should be recognized, stripped from E, phagocytosed, and destroyed by macrophages in the liver with subsequent recycling of CR1 deficient E. This indirect target clearance mechanism has some drawbacks that will be later discussed, while still demonstrating fast and somewhat efficient in vivo clearance in the circulation.

It is generally thought that the response to antigens involves both humoral responses and cellular responses. Humoral immune responses are mediated by non-cellular factors released by cells, which may or may not be found free in the plasma or intracellular fluids. A major component of a humoral response is mediated by antibodies produced by B lymphocytes.

Cell-mediated immune responses result from the interactions of cells, including antigen presenting cells and B lymphocytes (B cells) and T lymphocytes (T cells). The cellular immune system is comprised of cells of myeloid lineage, the polymorphonuclear granulocytes including neutrophils, basophiles, and eosinophils, and the monocytes both comprising the reticuloendothelial system (RES). The RES includes the immature circulating blood monocytes and the mature Kupffer cells in the liver, the cells of the intraglomerular mesangium of the kidney, the alveolar macrophages in the lung, the serosal macrophages, the brain microglia, spleen sinus macrophages and lymph node sinus macrophages. These phagocytic cells are characterized in Table III in terms of their surface receptors and their granular contents.

The immune response is initiated by the recognition of foreign antigens by various kinds of cells, principally macrophages or other antigen presenting cells leading to activation of lymphocytes, in particular, the lymphocytes that specifically recognize that particular foreign antigen and results in the development of the immune response, resulting in elimination of the foreign antigen. Overlaying the immune response directed at elimination of the foreign antigen are complex interactions that lead to helper functions, stimulator functions, suppressor functions and other responses. The power of the immune system's responses must be carefully controlled at multiple sites for stimulation and suppression or the response will either not occur, be over responded to or not continue after pathologic target elimination.

The recognition phase of response to foreign antigens consists of the binding of foreign antigens to specific receptors on immune cells. These receptors generally exist prior to antigen exposure. Recognition can also include interaction with the antigen by macrophage-like cells or by recognition by factors within serum or bodily fluids.

In the activation phase, lymphocytes undergo at least two major changes. They proliferate, leading to expansion of the clones of antigen-specific lymphocytes and amplification of the response, and the progeny of antigen-stimulated lymphocytes differentiate either into effector cells or into memory cells that survive, ready to respond to re-exposure to the antigen. There are numerous amplification mechanisms that enhance this response.

In the effector phase, activated lymphocytes perform the functions that may lead to elimination of the antigen and establishment of the immune response. Such functions include cellular responses, such as regulatory, helper, stimulator, suppressor or memory functions. Many effector functions require the combined participation of cells and cellular factors. For instance, antibodies bind to foreign antigens and enhance their phagocytosis by blood neutrophils and mononuclear phagocytes, free and fixed.

In general, the humoral immune system function results in the production of antibody specific to an invading immunogenic target and is mediated by T lymphocyte processing of the immunogen and transferring or presenting it to the B lymphocytes to initiate antibody production specific for the immunogen. The cellular immune system is comprised of cells of myeloid lineage, one, the polymorphonuclear granulocytes (neutrophils, basophiles, and eosinophils) and, two, the monocytes comprising the reticuloendothelial system (RES). All of the mature monocytes, due to their increase in size post migration into these tissues, remain fixed and cannot themselves reenter the circulatory system. They phagocytize a microbial invader or other immunogenic target post-modification that forms an opsonized immune complex and clearance of the opsonized immune complex from the body. Thus, the cellular immune defense in vertebrates has evolved to include antigen processing and antibody producing cells (lymphocytes) and macrophages of two distinct myeloid lineages. The resultant function of both systems is the clearance of any target from the body.

Role of Receptors in IC Clearance

Pathologic targets opsonized with antibody including their Fc region, with or without complement fixation, which results in the presence of the opsonin C3b on the IC, are more efficiently phagocytized by both the granulocyte and monocytes macrophages than in the absence of the opsonin. This is mediated by phagocytic cell Fc receptors (FcRs), specific for the Fc region of any antibody, and CR1 receptors specific for the major component of activated complement, C3b.

This phagocytotic clearance of the opsonized immune complex is known under appropriate conditions to function in both granulocyte and monocyte phagocytic cellular compartments. It is known by those skilled in the art that monocytes may live for months or years and comprise approximately 6% of normal WBCs, (White Blood Cells), and <2% of normal bone marrow cells. It is also known that granulocytes live 2 to 3 days and exist in the general circulation for 6 hours, comprise 60-70% of the total blood leucocytes and are produced in the bone marrow at a rate of 80 million per minute, and are released into the circulatory system.

As detailed in Table 1, immune complexes can be cleared from the body by natural mechanisms, including the direct interaction of the immune complex with the phagocytic cell both polymorphonuclear granulocytes (PMN) and monocytes, mediated by C3b and FcRs on the phagocytic cell surface (Table I-A and I-B), and the indirect interaction of the target with antibody sensitized erythrocyte (E) with the phagocytic cell, mainly the fixed tissue liver monocytes of the RES, mediated by binding of complement component (C3b) opsonized immune complexes to the CR1 found on the primate E (Table I-C).

Immune complexes may also be cleared by indirect artificial mechanisms: one, based on the CR1 exchange reaction for in vivo target clearance (Table I-D) involves the heteropolymer (HP=IgG anti target-IgG anti CR1) induced binding of the target to the HP sensitized primate Es possessing the E CR1 surface receptors which activate the CR1 exchange reaction and results in target clearance in the liver and spleen; and two, as proposed in the preferred embodiments of the present invention (Table I-E), target clearance by the indirect interaction of the target with a molecule pair sensitized RBC, with the MP represented as IgG anti target-Fab anti any site on the RBC other than CR1. This results in clearance of the E MP/target/C3b opsonized complex by a mechanism other than the CR1 exchange reaction involving multiple phagocytic compartments mainly in the liver and spleen and to some degree in the circulation.

The direct clearance of the immune complex from the blood stream normally is efficient, however, in some cases the clearance of an excess of IC often leads to immune complex deposition in tissues. This can lead to hypersensitivity in which subsequent complement activation causes an inflammatory response. This type of hypersensitivity is typically manifested as serum sickness, glomerulonephritis, rheumatoid arthritis and systemic lupus erythematosus.

However, the clearance of the immune complex by means of attachment to the CR1 receptor of primate Es, with the subsequent CR1 exchange reaction has been demonstrated to provide rapid and somewhat efficient target clearance from the circulation by phagocytosis of the target E HP complex by macrophage in the liver and spleen.

Immune complex clearance in the presence of activated complement component C3b immune complex (C3b) leads to a more efficient clearance mechanism based upon the presence of C3b or CR1 receptors on, the PMN phagocyte, on the monocytic macrophage and on the primate E. However, the presence of CR1 receptors on primate red blood cells competitively inhibits the PMN uptake and for the most part directs the immune complex (C3b) to the fixed monocytes in the liver and spleen for clearance by the CR1 exchange reaction.

The object of the present invention is the indirect clearance of the target by the E MP/target complex in the presence of complement activation functioning to attach C3b opsonin to the EMP/target complex (refer to Table I: E) and its clearance by phagocytic cell compartments.

Phagocytosis: The Programming of Indirect In Vivo Target Clearance

It is known by those skilled in the art that binding of the opsonized immune complex to erythrocytes (E) can lead to uptake and destruction of the erythrocyte-immune complex by phagocytosis. It is also known that the pathway and compartment selected for processing the erythrocyte-immune complex is dependent upon the number of immune complexes bound per erythrocyte and the homogenous surface distribution of available surface binding sites.

Once the target binds the primate E CR1 site, either directly or indirectly, it is cleared solely by passage primarily through the liver and secondarily through the spleen. In this scenario the circulating granulocyte phagocytic cell is excluded from the phagocytic clearance of the immune complex. It is known by those skilled in the art that the factor controlling compartmentalization of phagocytosis is the manner with which the immune complex interacts with the E. If the immune complex is attached to the CR1 site on E, it is precluded from granulocyte phagocytosis, known to be a result of the disperse patches of CR1 clusters on the E surface. The polymorphonuclear granulocytes for phagocytosis of the IC must recognize the even placement of the IC on the E generated by a homogeneous distribution of IC binding sites on the entire E surface; not provided by the CR1 disperse patches.

It is the object of the present invention that attachment of the IC at a site other than CR1 on the E allows the E IC complexes not only to be phagocytized in the liver and spleen, but also in the circulating PMN phagocytic compartment, thereby increasing the kinetics and overall efficiency of in vivo target clearance beyond that provided by the CR1 exchange reaction exclusively.

Rapid CR1 Surrogate Clearance of Immune Complexes from the Body: Use of a Heteropolymer for Complement Opsonized Independent, IC Clearance Via the CR1 Exchange Reaction (Table I-D)

A heteropolymer is defined as: a polymer comprised of two antibodies of differing specificity, one being the IgG anti-CR1 antibody (or any other antibody or antibody fragment with similar specificity), and the other being the IgG anti-pathologic target (microbe, etc.). The heteropolymer is used as a surrogate to replace C3b opsonization of the immune complex by directly attaching the immune complex to the CR1 site via the HP. This results in rapid movement of the target to the liver and spleen and in sequestration of the target in the phagocytic compartment of the liver and spleen monocytic macrophages, due to the binding of the target/HP complex to the “privileged” CR1 site. The following sequence of events will briefly present the E HP clearance of a pathogen:

1. E is sensitized, preferably in vivo, with a two-specific antibody pair, HP, such as the one described above.

2. E HP can interact by binding the pathologic microbe, and there is no requirement for complement fixation or activation.

3. E HP/microbe complex will travel to the liver and spleen in the circulation as a result of normal circulating function.

4. The complex will participate in the CR1 exchange reaction, which is triggered by an initial interaction of Fcγ receptors (FcγRs) on the Kupffer cell surface with the Fcγ regions of the E HP/target complex as well as with E HP (HP sensitized E only-no target present).

5. The HP/Target complex, and HP sans target will both simultaneously undergo the CR1 exchange reaction.

6. The E is released to the circulatory system deficient in the CR1 surface receptors the HP was attached to. The HP/target complex and HP alone is degraded post-CR1 exchange internalization by the fixed Kupffer cells or splenic fixed macrophage.

The CR1 exchange reaction, known to those skilled in the art, occurs primarily in the liver and spleen and can be described as follows:

Characterization of the HP CR1 Exchange Reaction

One, the E HP or E HP target complex, both sans complement bind to the FCγR on the hepatic and splenic fixed monocytes. Two, the binding triggers the release of a proteolytic enzyme that cleaves the CR1 moiety releasing the E deficient in CR1 back to the circulation and at the same time internalizing the HP or the HP complex (with pathologic target) for destruction. As such, as a result of the CR1 exchange reaction, CR1 numbers on the E surface are reduced.

Thus sensitized Es (E HP) in the absence of the target are themselves undergoing the CR1 exchange reaction and competitively inhibiting target clearance. The result of reduced numbers of CR1 sites on E, released back into the circulation, and the understanding that the CR1 receptor in normal Es has a limited expression on the surface, leads to an impairment of the total host immune response to other microbes not targeted by the HP or other soluble immune complexes, also not related to the targeted molecules. This generalized immune system depression although transient, remains a concern specifically in applications, which require repeated rounds of treatment with HP such as prolonged exposure to biological warfare agents or where the pathologic target is an autoimmune antibody in a chronic disease state.

A major drawback with the use of HP, in spite of its rapid clearance of pathologic targets, is the necessity to protect (“immunize”) the host long term from the pathologic target in numerous applications. In Table I, HP is represented rightly as affording limited protection.

It is also known that the rapid clearance mechanism of HP falls short of clearance of >99.9% of the pathologic targets. Data indicates that rapid clearance rates of large numbers of pathologic targets are demonstrated wherein no more than 90% of pathologic target clearance is achieved and the remaining 10% are not retrievable by this system, and the lost targets are considered sequestered in the vasculature system. Furthermore, it is also known that in primate model systems, use of mouse monoclonal antibodies manifests an immunologic reaction of monkey to the mouse antibody compromising the HP, resulting in decreased function of the HP in pathogen clearance by a mechanism resulting from complement opsonization rendering these E HPs unable to clear the pathologic target from the blood via this CR1 exchange pathway due to their inhibition.

Certain pathologic targets such as HIV and Marburg virus prove problematic for clearance with the CR1 exchange reaction possibly due to their being directed to the wrong phagocytic compartment, namely the fixed monocytes. Additionally, HIV virus pursues a low-grade infection in CD4 expressing monocytes. It may be quite important to direct both HIV and Marburg virus and other pathogens to a different phagocytic compartment for their destruction and clearance from the body.

The major impetus for use of the CR1 exchange reaction for pathologic target (microbial, chemical, and auto-antibody) is the rapid clearance of the pathologic target. In summary, problems with use of this strategy include:

Inability to retain E HP for a prolonged enough period (minutes),

Inability to clear all of the pathologic target load (only 90% attainable, with no current strategy to increase this to ˜100%),

Transient decrease in E CR1 which may compromise the body's natural complement opsonized clearance of pathogenic immune complexes by the E CR1 receptor and the CR1 exchange reaction.

The E HP/pathologic target is processed in a CR1 exchange reaction only in the liver (and to a lesser extent spleen) mediated by binding to the FcγR resulting in release of E with depleted CR1, possibly leading to organ toxification.

Similarly, the E HP sans pathologic target is similarly processed in a CR1 exchange reaction only in the liver (and to a lesser extent spleen) again mediated by binding to the FcR resulting in release of E with depleted CR1, in direct competition with clearance of the target/E HP complex,

Host immune reactions to the HP decrease the efficacy of the HP to function as designed especially after multiple HP injections.

CR1 depletion on the E surface, even though it may be transient at the time of pathologic target action, debilitates the host immune system at a critical period. This effect may be explained by the overwhelming of the Kupffer cells in the liver where processing an excess of E HP competitively inhibit the processing of the E HP/pathologic target complex. Furthermore, the E HP and subsequent CR1 reaction itself excludes the destruction of the target by the circulating blood phagocytic cells essentially limiting the total effectiveness of the host immune system. Lastly, cross species antibody rejection reactions activate complement which components C3b, C4b and the chemo-attractant C3a and C4a peptides produced mobilize the cellular immune system to the wrong target (namely, the E HP itself and not the pathologic target). Also, not all pathologic targets can be cleared using E HP from the bloodstream, due to the targets being sequestered in the vasculature, such as capillaries and small venules. Limiting the clearance reaction to the CR1 exchange reaction in the liver and spleen to fixed tissue macrophages (Kupffer cells) in the absence of complement activation makes this minor uncleared fraction of the pathologic target life threatening.

What is needed is a system of eliminating a pathologic target from the bloodstream that does not result in reduced immune system efficacy. The system used should protect or “immunize” the individual for a prolonged period, and be specific for applications such as prophylaxis for exposure to biological weapons and chronic, long-term, autoimmune disease. The system should be capable of clearing essentially >99.9% of the targets efficiently, wherever they are sequestered in the body. In preferred embodiments of the present invention, there may also be a fortuitous inclusion of the circulating PMN phagocytes in the clearance mechanism which may result in the increased target clearance over that observed in the liver and splenic monocytes.

### BRIEF DESCRIPTION OF THE TABLES

Table I depicts the clearance of immune complexes (IC) by direct and indirect methods. The direct methods involve the attachment of the opsonized (C3b bound) IC to phagocytic cells and its clearance. The indirect methods involve the attachment of the target to an antibody pair sensitized erythrocyte (E) (intact E or ghost E) with its subsequent clearance from the circulation.

Table II depicts a process comparison between heteropolymer (HP) CR1 exchange reaction IC clearance and molecular pair selective target elimination (STE) IC clearance with its three embodiments.

Table III details the surface receptors expressed in all the phagocytic cell compartments and their granular content.

Table IV lists the additional sites for possible attachment of the MP to the E surface.

Table V lists a glossary of terms used in the present application.

Table VI depicts the clearance of immune complexes (IC) by direct and indirect methods.

Table VII depicts a process comparison between heteropolymer (HP) CR1 exchange reaction IC clearance and molecule pair selective target elimination (STE) IC clearance with multiple embodiments.

### SUMMARY OF THE INVENTION

A method for elimination of pathological agents from the blood of a patient is described. The method of the present invention comprises administering to patient at least one sensitized erythrocyte having a molecule pair antibody complex that is capable of binding a pathological agent at a site other than the CR1 receptor, and eliminating the pathological agent from the patient's blood independent of the CR1 exchange reaction. The method includes wherein the molecule pair antibody complex comprises two antibodies that are covalently linked, wherein one of the antibodies is specific for binding to an erythrocyte receptor site and the other antibody is specific to the pathological agent. In one embodiment of this invention, the method includes wherein the antibodies are monoclonal antibodies. The method of this invention includes wherein the patient is a human being, non-human primate or an animal and wherein the antibodies are humanized or non-humanized antibodies. Preferably, the method of the present invention as described herein includes administering to the patient more than one sensitized erythrocyte having the molecule pair antibody complex.

In another embodiment of this invention, a method for blood-borne pathogen clearance in a patient in vivo is disclosed. This method comprises administering to a patient an effective amount of a molecule pair, wherein the molecule pair is prepared using humanized or non-humanized antibodies; allowing the molecule pair to bind to a specific immunogenic site on at least one erythrocyte surface different to CR1 thereby forming a sensitized erythrocyte molecule pair; and allowing the sensitized erythrocyte molecule pair to bind to a specific pathological target in the patient's blood to any site on the erythrocyte other than the CR1 resulting in an erythrocyte-molecule pair-pathological target, and clearing the erythrocyte-molecule pair-pathological target from the patient's blood.

Another embodiment of the present invention provides a method for blood-borne pathogen clearance in a patient in vivo comprising sensitizing at least one erythrocyte with a molecule pair ex vivo; administering an effective amount of the sensitized erythrocyte molecule pair to the patient; allowing the sensitized erythrocyte molecule pair to bind to a specific pathological agent resulting in an erythrocyte-molecule pair-pathological agent, and clearing the erythrocyte-molecule pair-pathological agent from the patient's blood.

A further embodiment of the present invention discloses a method for blood-borne pathogen clearance in a patient in vivo comprising preparing at least one erythrocyte ghost having senescence markers; sensitizing at least one of the erythrocyte ghosts with at least one molecule pair ex vivo; administering an effective amount of the sensitized erythrocyte ghost molecule pair to a patient; and allowing the sensitized erythrocyte ghost molecule pair to bind to a specific pathological agent present in the patient's blood resulting in an erythrocyte ghost-molecule pair-pathological agent, and clearing the erythrocyte ghost-molecule pair-pathological agent through the privileged apoptotic or senescent cell natural clearance system of said patient's body.

In yet another embodiment, the present invention provides a composition comprising an erythrocyte and a molecule pair antibody complex wherein said erythrocyte is capable of being bound to at least one of the molecule pair antibody complex at a site that is other than the CR1 receptor, and wherein the molecule pair antibody complex is capable of binding a pathological agent. In a further embodiment, this invention sets forth a composition comprising an erythrocyte ghost and a molecule pair antibody complex wherein the erythrocyte ghost is capable of being bound to at least one of the molecule pair antibody complex at a site that is other than the CR1 receptor, and wherein the molecule pair antibody complex is capable of binding a pathological agent.

### DETAILED DESCRIPTION OF THE INVENTION

As used herein the terms “patient” or “patients” means members of the animal kingdom, including such as for example but not limited to human beings, non-human primates, and animals.

The present invention provides a different indirect in vivo target clearance process from the above mentioned background art. The present applicants have coined the term Selective Target Elimination (STE) for describing the technology of the present invention. STE involves a number of embodiments that in general can be used for clearance of pathologic or other targets from the peripheral blood. These embodiments, STE I and STE II, intend to address the problems inherent to the HP clearance system.

In STE I (Table VI: E), a molecule pair (MP) always defined as IgG anti target-Fab anti any immunogenic site on the E surface other than CR1, is attached to the primate E forming E MP. Post MP injection, the sensitized E MP will rapidly bind the specific target in the circulation to any site on E other than the CR1 site resulting in phagocytosis of the E MP/target/C3b opsonized complex primarily in hepatic and splenic monocytes, and possibly including the circulating PMNs. The advantages of STE I are discussed herein.

STE II embodiments of the present invention are designed to improve E MP target clearance, wherein the MP ex vivo sensitizes erythrocyte ghosts (Eg). Post-transfusion into the body the Eg MP binds targets present in the circulation, and directs the pathologic target to the privileged apoptotic or senescent cell natural clearance system, utilized to clear trillions of apoptotic cells daily in a patient's body. STE II provides a short passive immunity period (STE IIa, Table VI: F) or a prolonged period of passive immunity (STE IIb, Table VI: G).

All of the aforementioned processes support efficient and rapid in vivo target clearance by activation of a naturally occurring process. E HP functions by utilization of the “privileged” CR1 exchange reaction. E MP STE I functions by utilization of the phagocytic cell surface receptors (PMNs and macrophages). Eg MP (STE IIa, STE IIb) function by the use of the natural apoptotic cell clearance mechanism in the bloodstream.

### Selective Target Elimination Strategies

The present invention involves a number of embodiments that in general can be used for clearance of pathologic or other targets from the peripheral blood. These targets may be microbes, toxic chemicals, toxins, autoimmune antibody and others. These embodiments, STE I and STE II, both address the problems inherent to the HP clearance system and its CR1 exchange reaction. STE embodiment designs address the downsides of HP clearance that are:

1. Use of a single phagocytic compartment, (all target in HP clearance is cleared only in the liver).

2. All targets, toxic or otherwise, are shunted to the liver risking organ toxification.

3. The clearance reaction is rapid and short lived (minutes).

4. Only 90% clearance is attainable (10% explained as being lost in the vasculature).

STE embodiments address these problems by attempting to add the circulating phagocytic compartment to the liver and spleen fixed tissue monocyte phagocytic compartments. This may provide the twofold effect of attaining better clearance rates, and minimizing the liver organ toxification risk in the HP system.

Embodiments of the current invention called Selective Target Elimination (STE) fall into two categories, herein, referred to as STE I and STE II. Both support in vivo pathologic target clearance independent of the CR1 exchange reaction. STE embodiments are presented in parallel with HP and CR1 clearance in Table II.

The E HP and E MP/Eg MP processes are presented herein and are characterized in terms of their overall benefits, downsides, and period of immunity conferred.

E HP: Use of the “Privileged” CR1 Site on the Primate E for Rapid in vivo Target Clearance in the Circulation Via the CR1 Exchange Reaction (Table VI: D and Table VII)

A heteropolymer is defined as a polymer comprised of two antibodies of differing specificity, one being the IgG anti-CR1 antibody and the other being the IgG anti-pathologic target. The heteropolymer is used as a surrogate to replace C3b opsonization of the immune complex by directly attaching the immune complex to the E CR1 site via the IgG anti-CR1. The following sequence of events will briefly describe the E HP clearance of a pathogen:

1. E is sensitized, preferably in vivo, with a two-specificity antibody pair, HP, such as one described above.

2. E HP interacts by binding the pathologic microbe, and no complement is required to be fixed or activated.

3. The E HP/target complex will travel to the liver and spleen in the normal circulation.

4. The CR1-HP-target grouping is stripped from E by the liver macrophages through a mechanism of clearance known as the Transfer Reaction by those skilled in the art. This reaction involves proteolysis of the E CR1.

5. The HP/Target complex, and HP sans target will both undergo the transfer reaction resulting in HP and HP/target phagocytosis and the removal of the E CR1 receptor.

6. The E is released to the circulatory system deficient in CR1 surface receptors.

Typically ≧95% of pathologic target clearance is achieved by using E HP. Since sensitized Es in the absence of the target are themselves undergoing the Transfer Reaction, this competitively inhibits the target clearance. The result of reduced numbers of CR1 sites on E, released back into the circulation, and the understanding that the CR1 receptor in normal Es has a limited expression on the surface, leads to an impairment of the host immune response to other targets not targeted by the HP or other soluble immune complexes, also not related to the targeted molecules. Also, the E HP clearance process would have limitations specifically in circumstances that would require repeated rounds of treatment with HP, such as prolonged exposure to biological warfare agents or where the pathologic target is an autoimmune antibody in a chronic disease state. HP will not provide long term protection to the host.

The use of mouse monoclonal antibodies on the HP manifests an immunologic reaction on the primate experimental model resulting from complement opsonization rendering these E HPs unable to clear the pathologic target from the blood via this CR1 exchange pathway due to HP damage.

In summary, problems with use of this strategy include:

Inability to retain E HP for a sufficient period (only minutes).

Transient decrease in erythrocyte CR1, which may compromise the body's natural complement opsonized clearance of pathogenic immune complexes by the E CR1 receptor and the CR1 exchange reaction.

The E HP/pathologic target is processed in a CR1 exchange reaction only in the liver (and to a lesser extent spleen) mediated by binding to the FcγR resulting in release of E with depleted CR1.

Similarly, the E HP sans pathologic target is processed in a CR1 exchange reaction only in the liver (and to a lesser extent spleen) again mediated by binding to the FcγR resulting in release of E with depleted CR1, in direct competition with clearance of the E HP/target complex.

Host immune reactions to the HP decrease the efficacy of the HP to function as designed especially after multiple HP immunizations.

Usual inability to clear >99% of pathologic target.

For applications such as prophylaxis for exposure to biological weapons, and chronic long-term autoimmune disease, what is needed is a system of eliminating a pathologic target from the bloodstream that does not potentially reduce immune system efficacy. The system used should also protect and provide passive immunity to the individual for a prolonged period, and it should be capable of clearing essentially >99.9% of targets efficiently, wherever they are sequestered in the body. We anticipate that STE clearance strategies support increased target clearance over that observed in HP-meditated clearance.

E MP: Use of the Natural Phagocytic Receptors for Rapid and Efficient Target Clearance Via Phagocytosis in Multiple Phagocytic Compartments not Involving the CR1 Exchange Reaction.

The present invention involves a number of embodiments that in general can be used for clearance of pathologic or other targets from the peripheral blood. These targets may be microbes, toxic chemicals, toxins, autoimmune antibody and others. Embodiments of the current invention called Selective Target Elimination (STE) fall into two categories, herein, referred to as STE I and STE II. Both support in vivo pathologic target clearance independent of the CR1 exchange reaction. STE embodiments of the present invention address the problems inherent to the HP clearance system by attempting to add the circulating phagocytic compartment to the liver and spleen fixed tissue monocyte phagocytic compartments, and also exploit other natural systems in the body to achieve improved target clearance. STE embodiments are presented in parallel with HP and CR1 clearance in Table VII.

Selective Target Elimination I (STE I)

STE I is characterized by addition of the circulating PMN phagocytic compartment to the macrophage compartment in the liver and spleen wherein target clearance would normally occur.

STE is characterized by the following upsides:

Provision of a 120 day passive immunity period based on the 60 day half-life of the primate E,

The inability to stimulate a host immune reaction to the immune globulin used conferring the passive immunity (for example, antibodies used are humanized),

The potential to neutralize and clear >99% of the pathologic targets present in the host possibly due to the expansion of the phagocytic compartments for target clearance,

The immediate neutralization and destruction of the pathologic target by complement fixation (complement trigger) prior to target clearance,

The clearance of target by phagocytosis of the circulating PMNs may improve that documented for the CR1 exchange reaction and macrophage involvement.

STE I involves the in vivo or ex vivo sensitization of the E with the MP. This method utilizes the intact circulating RBCs to indirectly clear the target present in the circulation.

The steps of STE I are:

Step I: E sensitization by MP: Injection in vivo of the MP, constructed as IgG pathologic target-RBC attachment antibody as described herein. The MP is composed of humanized mAbs, to avoid host immune reaction of the mAbs (initially of murine origin); and the target capture mAb is characterized as having an FC region suitable for complement fixation but incapable of being recognized by the FcγR on the liver and spleen fixed tissue monocytes and the PMNs in circulation. The MP sensitization of E may occur by injection of the MP in vivo or universal donor cell or autologous RBC sensitization by MP ex vivo followed by transfusion of the E MP.

In this system, the passive immunity provided by the injection possesses a 120 day duration, which is based upon the life expectancy of the RBCs. According to the tenants of this STE I embodiment, the E MP complex binds the target and fixes complement resulting in phagocytic clearance of the E MP/target complex by the PMNs and fixed tissue monocytes (present in the circulation and liver/spleen respectively).

Step II: Binding of E MP to the target resulting in complement fixation and activation.

Step III: Phagocytosis of E MP/target/C3b opsonized immune complex by the PMNs and macrophages.

Selective Target Elimination I (STE I):

STE I involves the in vivo or ex vivo sensitization of the E with the MP. This method utilizes the intact circulating red blood cells (RBC) to indirectly clear the target present in the circulation. The E is sensitized in vivo by injection of the MP into the body. Conversely, universal donor RBCs or autologous RBCs may be sensitized in vitro and the E MPs subsequently transfused into the body.

The MP is represented as IgG pathologic target-RBC attachment antibody fragment devoid of Fc region. The MP is composed of humanized Mabs to avoid host immune reaction against the Mabs (initially of murine origin), and the target capture Mab possesses a normal Fc region suitable for complement fixation; however this Fc region may need modification to avoid recognition by the FcγR on the liver and spleen fixed tissue monocytes and the PMNs in circulation. The circulating E MP immediately binds any pathologic target resulting in complement fixation and activation. The E MP/target/C3b complex is cleared form the circulation in a number phagocytic cell compartments including circulating PMNs, hepatic and splenic fixed tissue monocytes. The E MP sans target possesses no complement C3b opsonin allowing its longer term survival in the circulation.

STE I is characterized by addition of the circulating PMN phagocytic compartment for the clearance of the E/pathologic target complex along with the monocyte phagocytic compartment in the liver and spleen.

The STE II embodiment of the present invention employs RBC ghosts instead of intact RBCs, thereby avoiding the potential phagocyte toxicity of the RBC contents. While STE IIa is independent of complement activation, STE IIb possesses a complement trigger to initiate the Eg MP/target/C3b complex phagoctyic event.

Selective Target Elimination II (STE II) Embodiment One

Another STE embodiment of the present invention was designed to obviate the need for a complement trigger. This embodiment called STE II embodiment one can clear the same range of pathologic and other targets as STE I, however, the passive immunity is reduced to a shorter period such as for example, approximately an hour in contrast to 120 days for STE I.

The rationale for STE II is the transfusion of erythrocyte ghosts (Eg) sensitized with the MP which immediately binds the targets in vivo for clearance. Next, the transfused Egs are prior to target clearance preprogrammed to rapid phagocytosis and rapid clearance by any process that will allow the liver and spleen macrophages to recognize the Eg MP as an apoptotic cell. This is a natural clearance mechanism for apoptotic cells resulting in efficient binding of Eg MP membranes or mimic apoptotic cells to the macrophages and their subsequent internalization and destruction in the liver and spleen.

The mimic apoptotic cells, the Eg MPs, bind the target in vivo and are immediately cleared in the liver and spleen. Although STE I attempts to expand the phagocytic compartment to the circulating PMNs, such is not the aim of STE II. STE II uses the highly efficient apoptotic cell clearance system as a privileged mechanism for efficient in vivo target clearance just as the HP exploits the efficient CR1 exchange reaction for in vivo target clearance.

The Eg MP can be recognized and treated as a senescent apoptotic cell for clearance by the hosts natural apoptotic cell clearance mechanism by:

Chemically modifying E of all ages by addition of phosphatidylserine (PS) on the E surface before or after MP sensitization and subsequent E lysis.

Chemically modifying E of all ages by addition of Galactose α1,3 to human erythrocytes resulting in the creation of a senescence-associated epitope.

Lysis of MP sensitized E itself should result in the surface appearance of PS and render the Eg MP an apoptotic cell mimic,

Lysis of E MP in the presence of divalent cation (Mg⁺⁺) and in the absence of ATP results in high PS exposure on the Eg MP surface, whereas other methods without these chemicals provide ghosts with limited PS expression. The simultaneous presence of ATP during E lysis ex vivo will result in diminished PS exposure on the Eg surface.

Crosslinking of any RBC surface protein such as band-3 by hetero-bifunctional alkylating agents prior to MP sensitization and subsequent lysis to produce the Eg MP,

Isolation and collection of apoptotic RBCs by density gradient centrifugation, allowing only senescent RBCs to be sensitized and subsequently lysed to produce the necessary apoptotic mimic, Eg MP,

Any other physical or chemical treatment or other treatments known by those skilled in the art resulting in the production of the apoptotic mimic or natural apoptotic Eg MP.

The steps of STE II embodiment one are:

Step I: Sensitize universal donor RBCs, ABO type “O” or other autologous intact RBCs with the MP: IgG anti target-Fab anti any attachment site on the RBC other than CR1.

Step II: Treat the RBCs by a physical or chemical process, known by those skilled in the art, that will induce the sensitized RBC to become recognized as apoptotic. For example, this may include lysis of the intact E MP to produce Eg MP or any physical or chemical treatment known to those skilled in the art that will induce the apoptotic cell clearance mechanism by recognition of PS on the Eg MP surface. It is known that lysis of intact RBCs in the presence of divalent cations (Mg⁺⁺) results in the high level of expression of PS on the RBC ghost surface. It is also known that the level can be reduced by the concomitant addition of ATP to the lysis process which would allow the translocase enzyme to actively bury the surface PS between the membrane layers, thus offering a surface PS modulation mechanism. It is known to those skilled in the art that apoptotic RBCs are phagocytized in a natural mechanism by the monocyte phagocytic compartments.

Step III: The target-specific MP sensitized apoptotic mimic RBCs (Eg MPs) are transfused into the host, whereupon, the targets immediately bind to the Eg MPs. This is supported by studies in the E HP system, indicating rapid binding of the targets in a few minute period to the E HPs upon HP injection.

Step IV: The mimic apoptotic state of the Eg MP is characterized by the movement of phosphatidylserine (PS) from deep membrane layers to the membrane surface. It is known to those skilled in the art that this induces efficient macrophage phagocytosis of the Eg MP by the natural mechanism.

In the STE II embodiment one, the trigger for the clearance method is the transfusion of induced apoptotic mimic MP RBCs and their clearance. The target to be cleared is bound by the MP specific molecule pair on the Eg surface and cleared. The binding of the target by the MP often will neutralize a toxin or the toxicity of a poisonous chemical, until the target/Eg MP can be ingested and cleared by the macrophages.

The STE II embodiment one necessitates the use of intact MP sensitized RBCs with highly exposed PS on the E surface, resulting in immediate clearance of the mimic apoptotic E. It is known to those skilled in the art that the contents of an intact ingested RBC will cause decreased phagocytosis in the RBC ingested macrophage. To avoid this unwanted reaction, the E MP apoptotic mimic may be lysed by any method known to those skilled in the art and E MP ghost membranes are isolated. The ghosts sans cytoplasmic contents post transfusion are efficiently cleared by the liver and spleen monocytes.

STE II embodiment one is characterized by:

A possible increase in the number of phagocytic compartments,

Short term passive immunity,

Inability to stimulate a host immune reaction to the immune globulin conferring the passive immunity,

The potential to clear >99% of the pathologic targets present in the host,

The lack of dependence on complement fixation or activation for the process to occur,

A rapid rate of clearance of the pathologic target, similar to that observed in the CR1 exchange reaction (minutes).

Selective Target Elimination II (STE II) Embodiment Two

Another STE embodiment of the present invention is designed to function with a complement trigger. This embodiment called STE II embodiment two can clear the same range of pathologic and other targets as the other STE embodiments, however, the passive immunity is prolonged for a lengthy period such as for example, months.

In STE II embodiment one, the high Eg surface expressing PS level functions to preprogram the Eg MP for rapid clearance by the apoptotic cell clearance pathway, and the period of immunity is short-lived. STE II lengthens the period of passive immunity to months.

In STE II embodiment two the Eg surface PS is neutralized or effectively “buried” by any mechanism known to those skilled in the art, including binding of annexin V, IgG anti PS, or MP (IgG anti pathologic target-Fab anti PS) and any other similar mechanism blocking Eg MP surface PS recognition by the PS receptor on the macrophage surface.

The steps of STE II embodiment two are:

Step I: Sensitize intact universal donor RBCs, ABO type “O” or autologous intact RBCs with the MP:IgG anti target-Fab anti any attachment site on the RBC other than CR1.

Step II: Lyse the E MP by any method resulting in varied surface expression of PS on the Eg MP surface. Since the object of this embodiment is to prolong survival of the Eg MP in the circulation, the PS sites present on the Eg MP surface can be neutralized as described herein. Binding an additional MP to the Eg MP, namely IgG anti target-Fab anti PS will prevent macrophage recognition of the apoptotic cell mimic, the Eg MP.

Step III: Bind the target for clearance to the Eg MP thereby activating the complement trigger by the opsonization of C3b to the Eg MP surface. This C3b will be the only signal to induce Eg MP phagocytosis by the natural mechanism in fixed monocytes in the liver and spleen. The antibodies of the MPs used herein will be humanized and possess a modified Fc region not recognized by the Fcγ receptor in macrophages, adding to the in vivo survival of the Eg MP.

Step IV: Clearance of the Eg MP/target/C3b opsonized complex by the macrophages in the liver and spleen.

STE embodiment two is characterized by:

A possible increase in the number of phagocytic compartments

Long term passive immunity

Inability to stimulate a host immune reaction to the immune globulin conferring the passive immunity

The ability to clear >99% of the targets present in the host

The presence of a complement trigger

The rapid and continuous clearance of the specific target post target appearance in the host circulation.

Selective Target Elimination IIa (Eg MP): Use of the Natural Apoptotic Cell Clearance Mechanism for in vivo Clearance of Targets Present in the Circulation (Short Term Passive Immunity).

The RBC has a life span of 120 days. As they become senescent, changes in membrane structure and integrity occur, such as phosphatidylserine (PS) exposure on the outer leaflet of the membrane and Band 3 clustering, among others as understood by those skilled in the art. Those changes signal the RBC removal from the circulation and promote macrophage-mediated erythro-phagocytosis in the spleen and liver. This is a natural clearance mechanism occurring in the body for clearance of RBC senescent cells. It is estimated that 360 millions of RBCs are phagocytized every day.

Based on this mechanism, for the STE IIa process we prepare RBC ghosts, generate the senescence markers on the ghosts and sensitized them with the MP (Eg MP). The rational for STE IIa action is the transfusion of Eg MP which immediately binds the targets in vivo. The Eg MP/target complex is immediately recognized as a senescent cell for clearance, in the spleen and liver, through the natural apoptotic/senescent cell clearance pathway.

Although STE I attempts to expand the phagocytic compartment to the circulating PMNs, such is not the aim of STE IIa. STE IIa uses the highly efficient apoptotic cell clearance system as a privileged mechanism for efficient in vivo target clearance just as the HP exploits the efficient CR1 exchange reaction for in vivo target clearance.

The Eg MP can be recognized and treated as a senescent apoptotic cell for clearance by the body's natural mechanism by:

Chemically modifying E of all ages by addition of phosphatidylserine (PS) on the E surface before or after MP sensitization and subsequent E lysis.

Lysis of E in a hypotonic solution itself should result in the surface appearance of PS and render the Eg MP an apoptotic cell mimic.

Lysis of E MP in the presence of divalent cation (Mg++) and in the absence of ATP results in high PS exposure on the Eg MP surface, whereas lysis with ATP provides ghosts with limited surface PS expression.

Crosslinking of RBC surface protein such as band-3 by hetero-bifunctional cross-linking reagents as known by those skilled in the art, or antibody cross-linking prior to MP sensitization and subsequent lysis to produce the Eg MP.

Isolation of apoptotic RBCs by density gradient centrifugation, allowing only senescent RBCs to be sensitized and subsequently lysed to produce the necessary apoptotic mimic, Eg MP.

Any other physical/chemical treatment or other procedures known by those skilled in the art resulting in the production of the apoptotic mimic or natural apoptotic Eg MP.

In STE IIa the trigger for the clearance mechanism is the transfusion of induced apoptotic mimic Eg MPs. There is no complement trigger to initiate the apoptotic cell clearance; however, it is known by those skilled in the art that both the classical and/or the alternate pathway participate in a later stage of the clearance process. In STE IIa the target to be cleared is bound by the MP specific molecule pair on the Eg surface and cleared with the ghost. The binding of the target by the MP often will neutralize a toxin or the toxicity of a poisonous chemical, until the target/Eg MP can be ingested and cleared by the macrophages.

STE IIa is characterized by:

Short term passive immunity,

Inability to stimulate a host immune reaction to the immune globulin conferring the passive immunity,

The ability to clear >99% of the pathologic targets present in the host,

The lack of a complement trigger to initiate clearance.

A rapid rate of clearance of the pathologic target by an efficient natural mechanism.

Selective Target Elimination IIb (Eg MP): Long Term Passive Immunity

In STE IIa the high Eg surface expressing PS level functions to preprogram the Eg MP for immediate clearance by the apoptotic cell clearance pathway, and the period of immunity is short-lived. To lengthen the period of passive immunity to possibly months STE IIb was designed.

In STE IIb the Eg is prepared having low or no PS surface exposure. PS is neutralized or effectively “buried” by any mechanism known to those skilled in the art, including binding of annexin V, IgG anti PS, or MP (IgG anti pathologic target-Fab anti PS), or any other mechanism, which effectively blocks the Eg surface PS from recognition by the macrophage PS surface receptor.

The Eg is next sensitized with the MP specific for the target to be cleared. Since it is known that PS is recognized by the PS receptor on the macrophage surface and provides the initial site of phagocyte attachment to the Eg MP, burying the PS would support prolonged survival of the Eg MP in the circulation, whereupon the targets marked for clearance are bound forming the Eg MP/target complex. Upon complex formation, complement is fixed and the Eg MP/target/C3b complex is phagocytized by the macrophages through the CR1 scavenger receptor on the macrophage surface. Herein, the C3b will be the sole signal to induce target complex phagocytosis. The antibodies of the MP will be humanized and may possess a modified Fc region to avoid recognition by the FCγR on the macrophages in the liver and spleen, adding to in vivo survival of Eg MP.

STE IIb is characterized by:

An increase in the number of phagocytic compartments.

Long term passive immunity.

Inability to stimulate a host immune reaction to the immune globulin conferring the passive immunity.

The ability to clear >99% of the targets present in the host.

The presence of a complement trigger.

The rapid and continuous clearance of the specific target.

STE IIb′ (Eg MP): Another Embodiment for Use of the Natural Apoptotic Cell Clearance Mechanism for Prolonged in vivo Clearance of Targets Present in the Circulation.

STE IIb′ embodiment of the present invention combines the characteristics of STE IIa and IIb. RBC ghosts are prepared to promote aggregates of the band-3 polypeptide, a major RBC membrane protein. It is well known by those skilled in the art that aggregation of band-3 generates neo-antigens recognized by natural auto-antibodies present in the host circulation. Furthermore, phagocytosis of damaged RBCs by the macrophages in the liver and spleen is mediated by the antibody binding to clustered band 3 antigen and activation of the alternative complement pathway as understood by those skilled in the art.

It is also known that RBC infected with Plasmodium (IRBC), the parasitic agent of Malaria disease, present membrane alterations such as clustering of the band 3 protein promoting the RBC clearance through the phagocytic compartments. Moreover, it is known that anti-malarial drugs considerably reduce the binding of the auto-antibodies to band 3, resulting in the failure of IRBC phagocytosis.

Although anti-malaria drugs produce some minor side effects, they are recommended as prophylaxis for travelers to Malaria endemic areas. From a practical standpoint to secure a strong response against any pathological target there would not be any restriction for use of this type of pharmacologic substance.

In the STE IIb′ embodiment of the present invention, the use of MP sensitized RBC ghosts characterized by clustering of band 3 and low to no PS surface exposure, co-administered with anti-malaria drugs promotes in vivo survival of the Eg MP. The clearance signal for the Eg MP is provided by the band-3 crosslinking after blood levels of the drug have been allowed to diminish.

STE IIb′ embodiment is then characterized by:

Long term passive immunity

Inability to stimulate a host immune reaction to the immune globulin conferring the passive immunity

The ability to clear >99% of a range of targets in the host

Rapid and continuous neutralization of the specific targets

Possible Redirection or Inclusion of Additional Phagocytic Compartments for the Clearance of Immune Complexes in Primates: Use of the Molecule Pair in STE I and STE II

An object of the present invention is to provide processes for the efficient and safe clearance of any pathologic target, such as an invading microorganism or an autoimmune antibody, from the bloodstream by a mechanism differing from the CR1 exchange reaction. There is a need to resolve the problems of immune complex clearance by the CR1 exchange reaction and its subsequent depletion of erythrocyte CR1 sites and phagocytosis of the E CR1/pathologic target complex exclusively in the liver and spleen by fixed phagocytic monocytes.

The factor that controls the granulocyte vs. fixed monocyte clearance of the immune complex is the site of attachment of the immune complex to the E. Attachment of the immune complex to the E CR1 site, due to its presence in discrete and limited numbers of patches on the E surface, directs the E immune complex to the monocytic macrophage fixed in the liver and spleen, where the CR1 exchange reaction occurs. However, attachment of the immune complex to any other site, besides CR1 on the E surface, mediated by an MP, due to the homogeneous dispersion of these protein attachment sites, may shift the clearance to the circulating PMN granulocyte phagocytes. Table III presents a list of additional possible sites for MP attachment to the E surface. These sites are carbohydrate and protein, all immunogenic in nature, and are expressed on the E surface. The CR1 site has limited expression on the E surface and as such shunts E HP immune complexes to the fixed tissue macrophages and CR1 exchange. In STE, the entire E MP/pathologic target complex is phagocytized completely only if multiple immune complexes bind over the entire E surface, each resulting in activation of pseudopod extension over a limited surface area, and many immune complexes binding over the entire E surface, support the total ingestion and destruction of the E and all its bound immune complexes. Phagocytosis has been described as a zipper mechanism, where the phagocytic compartment the E IC is directed to may include the PMN circulating granulocyte.

Use of the molecular pairs (MPs), MP (a₁a₂), used for immunogen or microbe clearance from the blood, or MP (a-ag), used for autoimmune antibody clearance from the blood directs the attachment of the immune complex, primarily, away from the CR1 site and clearance by the exchange reaction and the fixed monocytes in the liver and spleen to any other surface expressed immunogenic molecules on the E surface and to clearance by a number of phagocytic cell compartments via phagocytosis of the E MP/target complex.

A preferred embodiment of the present invention E MP (a₁a₂) is an antibody pair, namely one antibody specific to the Rho (D) site on the primate or human erythrocyte covalently linked by any method known to those skilled in the art to another antibody specific for the pathologic target.

The following chart explains the method:

THE MP (a₁a₂) CONSTRUCT IN Rh POSITIVE PEOPLE

| | | ABILITY |
|---|---|---|
| | | TO |
| | FUNCTION | FIX C′ |
| a₁ | Attachment to E at Rho (D) locus or other site (other than | NO |
| | CR1) | |
| a₂ | Capture of pathologic immunogenic target to be cleared | YES |

The antibodies may be of any type or an antibody fragment (Fab)₂ or Fab devoid of the Fc region, or the site of attachment of the antibody pair to the E may be in other embodiments at any surface protein or carbohydrate that is homogeneously expressed on the E surface via the corresponding specificity antibody, excluding CR1. Table III presents possible sites of attachment of the MP to the E surface.

Another preferred embodiment of the present invention includes an antibody-antigen (a-ag) pair, wherein the attachment antibody in the preferred embodiment is similar to that presented in the a-a pair, namely an anti Rho (D) antibody or antibody fragment covalently attached to an antigen for rapid removal of the antibody specific for the antigen in the host. Again, in other embodiments the site of attachment of the a-ag pair to the E surface may be any protein or carbohydrate that is homogeneously expressed on the E surface with use of the corresponding specificity antibody excluding the CR1 site on E.

THE MP (a-ag) CONSTRUCT IN Rh POSITIVE PEOPLE

| | | ABILITY |
|---|---|---|
| | | TO |
| | FUNCTION | FIX C′ |
| a | Attachment to E at Rho (D) locus or other site (other than | NO |
| | CR1) | |
| ag | Capture of pathologic antibody target to be cleared | YES |

Approximately 10-20% of people worldwide are Rhₒ negative and do not possess the D antigen on their cell surface. Two other preferred embodiments of the present invention involve the attachment of the sensitizing pairs to another protein homogeneously expressed on the entire RBC surface. Table III provides a list of potential binding sites.

To attach the a-a pair or the a-ag pair to the Es of Rh negative individuals another method will be employed, namely the use of an antibody, more specifically, an antibody fragment either an (Fab)₂ or Fab to anchor the pairs to the E surface. Lack of the Fc region on the attachment antibody will similarly block complement opsonization of the sensitized E and supports the E MP (a-a) or E MP (a-ag) sensitized E long-term survival (up to 120 days) due to the modified antibody being devoid of Fc region thereby being refractory to the FcγR receptor on fixed monocytes in the liver. The absence of the Fc region on the anchor antibody of all MP pairs, will similarly prevent complement fixation and activation at the Rhₒ (D) MP attachment site. The following chart explains some of the preferred embodiments:

THE MP (a₁-a₂) CONSTRUCT IN Rh NEGATIVE PEOPLE

| | | ABILITY |
|---|---|---|
| | | TO |
| | FUNCTION | FIX C′ |
| a₁ | Attachment of antibody fragment at any site | NO |
| | homogeneously expressed of the E surface other than | |
| | CR1 devoid of Fc | |
| a₂ | Capture of immunogen to be cleared from the | YES |
| | bloodstream | |

THE MP (a-ag) CONSTRUCT IN Rh NEGATIVE PEOPLE

| | | ABILITY |
|---|---|---|
| | | TO |
| | FUNCTION | FIX C′ |
| a | Attachment of antibody fragment at any site | NO |
| | homogeneously expressed of the E surface other than | |
| | CR1 devoid of Fc | |
| ag | Capture of antibody to be cleared from the bloodstream | YES |

In preferred embodiments of the present invention, the MPs are defined as:

MP (a₁-a₂) PAIR

Rh POS E

E/IgG anti Rhₒ (D)-IgG anti pathologic target

E/(Fab)₂ or Fab anti other surface protein other than CR1-IgG anti pathologic target

Rh NEG E

E/(Fab)₂ or Fab anti other surface protein other than CR1-IgG anti pathologic target

MP (a-ag) PAIR

Rh POS E

E/IgG anti RHO(D)-antigen specific for autoimmune Ab

E/(Fab)₂ or Fab anti other surface protein other than CR1-antigen specific for autoimmune Ab

Rh NEG E

E/(Fab)₂ or Fab anti other surface protein other than CR1-antigen specific for autoimmune Ab

In preferred embodiments of the present invention:

None of the above sensitized E fix complement (prior to pathologic target binding).

Complement is fixed and activated post pathologic target binding only, which triggers phagocytosis.

All the above sensitized Es will be resistant to phagocytosis due to drug or other mechanism induced, decreased expression of the FcγR on the phagocytic cells, both granulocytic and monocytic surfaces.

Another embodiment of the present invention provides for the achievement of maximal E MP survival by genetically engineering an antibody possessing an Fc region capable of fixing complement, the target capture antibody, that is not recognized by the FcγR receptors in the liver and spleen. A preferred aspect of two embodiments previously mentioned for E possessing the D surface antigen is the use of the common anti Rhₒ (D) antibody to anchor either the antibody [anti Rhₒ (D)]-antibody (anti pathologic target) or the antibody [anti Rhₒ (D)]-antigen (specific for a pathologic autoimmune or other antibody) to the E surface in such a manner that there is a homogeneous dispersion of these pairs on the E surface in order to encourage granulocytic phagocytosis. Characteristic of both these pairs is that, one, attachment of the anti Rhₒ (D) antibody to the E Rho (D) proteins does not fix or activate immune complement, and, two, that the homogeneous dispersion on the E surface of both MP pairs will, upon interaction with the respective pathologic target, fix and activate complement and stimulate phagocytosis by phagocyte complement opsonin receptors.

Production of the sensitized Es, namely E MP (a₁-a₂) and E MP (a-ag), are unable to fix complement, by design, in the Rh positive and negative host. These sensitized Es, themselves prior to attachment of the pathologic target, are susceptible to clearance from the bloodstream if Fc regions are present that will interact with the FcγRs that are known to be located on all phagocytic cells, PMNs and monocytes. This is illustrated in the following chart:

| | | |
|---|---|---|
| | | Cleared via |
| Fix Complement | Possess | FcγR on |

| | Target | Target | Fcγ | phagocytic |
|---|---|---|---|---|
| | absent | present | Regions | cells |
| Rh Positive | | | | |
| Host | | | | |
| E MP (a₁a₂) | NO | YES | YES | YES |
| E MP (a-ag) | NO | YES | YES | YES |
| Rh Negative | | | | |
| Host | | | | |
| E MP (a₁a₂) | NO | YES | YES | YES |
| E MP (a-ag) | NO | YES | NO | NO |

Inhibition of the Fc Mediated Clearance of E MP Prior to Binding of Their Pathologic Targets

E MPs upon proper construction may remain in the circulatory system for a maximum period of 120 days, which represents the 60-day half-life of an erythrocyte. It is known by those skilled in the art that granulocytes and fixed macrophages, including the Kupffer cells in the liver, possess surface FcγRs that attach immune complexes possessing normal Fc regions, such as E MP (Fc). It has been established that the phagocytic reaction occurs in two stages, the attachment of the Fc expressing immune complex to the Fcγ receptor, which then triggers the local pseudopod engulfing reaction. In order to phagocytize the entire E immune complex, multiple Fc determinants must be bound over the entire E surface. In preferred methods, this reaction is blocked by any means so that the E MPs will not be cleared from the bloodstream prior to binding the pathologic target.

The E MPs do not fix complement and three of the four embodiments presented possess Fc regions, methods are employed to block the interaction between the E MPs and the FcγR sites located on all phagocytic cells.

It is known to those skilled in the art that numerous methods exist to interfere with the E MP Fc region interaction with the FcγR. A preferred method for the present invention is the use of sex hormones, which are known to exert an effect on autoimmune disorders and immune cytopenia. One effect of sex hormones is to decrease the expression of the FcγRs on all phagocytic cells. It is known that glucocordicoids, progesterone, and androgen, excluding danozol, decrease the expression of the FcγRs on phagocytic cells. It has further been demonstrated that:

Androgens primarily reduced surface expression of FcγR1, 2 and FcγR2,

Drugs reversing the effect of androgens had no effect on macrophage FcγR expression,

These androgen-reversing drugs counteracted the effects of androgens on macrophage FcγR expression,

Both FcγR1, 2 and FcγR2 are expressed on all granulocytic and splenic macrophages and function to bind the E immune complex (Fc) to the macrophage.

This, combined with knowledge that FcγR decreased expression has no effect on immune complex (C3b) recognition by C3b (CR1) receptors on the macrophage surface and its subsequent phagocytosis. E MP/target complexes will fix and activate complement and be cleared by any phagocytic cell expressing the C3b surface receptor.

Another method used to negate the effect of the FcγR receptors includes the introduction of excess soluble Fc region to the system that would competitively inhibit the reaction of the E MP with the FcγR.

Lastly, as previously stated, the Fc domains responsible for complement fixation and FcγR recognition map to different loci. A recombinant Fc fragment may be constructed that will support efficient Clq binding (complement fixation), and subsequent complement activation, without being recognized by the FcγR receptor on macrophage surfaces.

It is known to those skilled in the art that modification of the FcγR will prolong E MP and Eg MP survival in the host circulation. It is also the object of STE to extend the target clearance form the macrophages in the liver and spleen to include the circulating PMN phagocytes. Those skilled in the art understand that the FcγR III mediates neutrophils recruitment to phagocytize immune complexes. An Fc modified region to avoid binding of the E MP or Eg MP to the FcγR on the liver and spleen macrophage may similarly preclude binding of the E MP or Eg MP to the PMNs. In this scenario, a complement trigger will support the required phagocytosis of the E MP/target/C3b and Eg MP/target/C3b complexes in vivo.

Combined Injection of MP and Androgen In Vivo Provides Optimal Longevity for the E MP Complex

It is known to those skilled in the art that immune complex (Fc) and E immune complex (Fc) are cleared by a mechanism mediated by the FcγR on the phagocytic cell surface. A similar phenomenon, in vivo, will occur in the presence of E HP and similarly E MP. Combined administration, in vivo, of MP and androgen or any other similarly acting substance known to those skilled in the art or genetic modification of the Fc region to non-recognition by the FcγR on the phagocytic cell surface, will result in protection of E MP and a decreased expression of FcγRs on all phagocytic cells. This will support a mean survival time of E MP in vivo, of 120 days, and is reversed by introduction of a drug to counteract the androgen effect at any time, or continued by repeated administration at prescribed intervals.

Complement Fixing and Activating Capability of E MP/Target Complex In Vivo

The E MP/Target complexes to be cleared from the bloodstream fix and activate complement. The activation of complement and the subsequent C3b opsonization of the E MP/Target complex are necessary to initiate phagocytosis of the E MP/Target/C3b complex by circulating granulocytes. This may be mediated by the presence of C3b receptors expressed on the surfaces of the granulocyte phagocytes. Similar C3b receptors in the fixed liver and spleen monocytes also participate in this clearance.

Kinetics of E MP Clearance of a Pathologic Target by STE I

The following steps occur upon injection of the specific MP for the target to be cleared and the Rhₒ (D) type of the host, which has been modified to be refractory to the FcγR receptors to avoid premature clearance of the E MP prior to target attachment.

The E possessing numerous MPs on its surface will remain in the bloodstream and active for up to 120 days (and can be extended by additional injection).

Each E MP binds a single pathologic target to be cleared.

This initial binding is followed by additional binding of pathologic targets over the E MP surface

Complement fixation of the E MP/Target complex allows the first target attached to anchor the E MP/Target complex to the macrophage and PMN, and pseudopods will partially engulf the E.

This E MP/Target/Macrophage complex continues to circulate in an intact state until sufficient pathologic target is bound and complement is activated to support complete erythrocyte phagocytosis.

Simultaneously complement fixation by the target and the E MP leads to immediate destruction of some microbial targets by the mechanism of complement fixation and activation in the classical complement pathway and the alternate complement pathway, known to those skilled in the art.

Complete phagocytosis is dependent on the binding of a number of targets to the E MP and this continues to occur while the E MP is attached to the circulating phagocytic cell.

The rate of clearance of the pathologic target is less important, with use of E MP than in the use of E HP and CR1 exchange reaction to clear targets, where as little as a single target on an E can be cleared by E HP and the pathologic target remains viable, thereby requiring rapid clearance, and assuming all the liabilities which such that have been previously stated.

Using E MP, once a minimum threshold of complement inactivated targets are attached to the E MP/Target/PMN complex, the PMN can completely internalize and destroy the MP/target complexes, and the E MP/target complex may also be attached to fixed tissue macrophages in the liver and spleen and cleared.

This extended immunity to the pathologic target supports applications such as in vivo neutralization of weapons of biological warfare, as well as continuous long-term clearance of autoimmune antibody in patients with this disease state.

Kinetics of Eg MP Clearance of a Pathologic Target by STE II Embodiment One

The Eg MP in this embodiment possesses a large number of PS sites on the ghost surface.

The transfused Eg MP immediately binds the pathologic target if present in the circulation.

The exposed PS binds to the PS receptor on the fixed tissue monocytes of the liver and spleen, where they are immediately cleared due to their recognition as scenescent apoptotic cells.

The duration of Eg MP in the circulation in this embodiment is limited to a period of hours.

No complement fixation is necessary to trigger phagocytosis by this natural apoptotic cell clearance pathway, however, PS exposed on the ghost erythrocyte surface has been shown to activate the alternate complement pathway and result in deposition of C3b onto the Eg MP. This may explain the rapid nature of the apoptotic cell clearance pathway.

Kinetics of Eg MP Clearance of a Pathologic Target by STE II Embodiment Two

The Eg MP possesses a small number of PS sites on the ghost surface. The few PS sites present will be “buried” by complexation with the MP (IgG anti target—IgG anti PS) preventing macrophage recognition of the Eg MP and its prolonged survival in the circulation.

The transfused Eg MP immediately binds the pathologic target if present in the circulation.

The inability of the Eg MP itself to trigger phagocytosis due to blocking of surface PS sites and modification of the Fc regions on the antibody present prolong the Eg MP survival in the circulation for months.

Complexation of the target with the Eg MP subsequently results in complement fixation and the opsonization of the Eg MP/target complex with C3b.

The C3b generated by a complement trigger marks the Eg MP/target complex for clearance by the fixed monocytes of the liver and spleen mediated by their surface C3b receptors.

The clearance of the target similarly continues in the body as long as the Eg MP exists in the circulatory system.

Whereas, particular embodiments of this invention have been described herein for the purposes of illustration, it will be evident to those skilled in the art that numerous variations of the details of the present invention may be made without departing from the invention as defined in the appended claims.

TABLE I

| | | | | | | DURATION |
|---|---|---|---|---|---|---|
| | | | | | | OF |
| | | | | | | FUNCTION |
| | | | CLEARANCE | | | OF |
| | CLEARANCE | | MEDIATED | SITE OF | RATE OF | PROTECTIVE |
| | OF | OPSONIZATION | BY | CLEARANCE | CLEARANCE | SYSTEM |
| A | Immune | w/o C3b | Phagocytic | Possibly | Slow | Lifetime |
| | complex | IgG only | cells | PMN in | | |
| | | | | circulation | | |
| | | | | primarily | | |
| | | | | monocyte | | |
| | | | | spleen and | | |
| | | | | liver | | |
| B | Immune | IgG, C3b | Phagocytic | Possibly | More rapid | Lifetime |
| | complex | | cells | PMN in | | |
| | | | | circulation | | |
| | | | | primarily | | |
| | | | | monocyte | | |
| | | | | spleen and | | |
| | | | | liver | | |
| C | Immune | IgG, C3b | E CR1 | Exclusively | Very rapid | Lifetime |
| | complex | | (CR1 | monocytes | | |
| | | | exchange | spleen and | | |
| | | | reaction) | liver | | |
| D | Immune | IgG only | E HP | Exclusively | Very rapid | Minutes to |
| | complex | (C3b not | (CR1 | monocytes | | hours |
| | | required) | exchange | spleen and | | |
| | | | reaction) | liver | | |
| E | Immune | IgG, C3b | E MP | Possibly | More rapid | 120 Days/ |
| | complex | | Phagocytosis | PMN in | | can be |
| | | | | circulation | | extended |
| | | | | primarily | | |
| | | | | monocytes | | |
| | | | | spleen and | | |
| | | | | liver | | |

TABLE II

| | | STE II | STE II | |
|---|---|---|---|---|
| Process | | Embodiment | Embodiment | |
| Characterization | STE I | One | Two | HP CR1 |
| Globulin type | Antibody Pair | Antibody Pair | Antibody Pair | Antibody Pair |
| (RBC attachment | MP (molecule | MP (molecule | MP (molecule | HP (hetero- |
| and target capture | pair) | pair) | pair) | polymer) |
| antibodies) | | | | |
| RBC | All sites other | All sites other | All sites other | CR1 only |
| Attachment | than CR1 and | than CR1 and | than CR1 and | |
| site | artificial site | artificial site | artificial site | |
| Attachment | Fab, (Fab)₂, | Fab, (Fab)₂, and | Fab, (Fab)₂, and | IgG anti CR1 |
| antibody | (devoid of Fc) | IgG with | IgG with | only (normal |
| | incapable of | normal Fc (fix | normal Fc (fix | Fc) |
| | fixing | complement) | complement) | |
| | complement | | | |
| Pathologic target | IgG (must | Fab, (Fab)₂, IgG | Fab, (Fab)₂, IgG | Fab, (Fab)₂, |
| capture antibody | possess Fc) or | (normal Fc) | (normal Fc) | IgG |
| | modified Fc to | | | (normal Fc) |
| | fix complement | | | |
| Globulin delivery | Injection of MP | Ex vivo RBC | Ex vivo RBC | Injection of HP |
| method | of transfusion | ghost (high | ghost (low | |
| | of E MP | surface PS) | surface PS) | |
| | | sensitization of | sensitization of | |
| | | universal donor | universal donor | |
| | | cells, type O, | cells, type O, | |
| | | and their | and their | |
| | | transfusion | transfusion | |
| Duration of | Long-lasting | Intermediate | Long-lasting | Short-lived |
| passive immunity | | lasting | | |
| period | | | | |
| Other antibody | Humanized | Humanized | Humanized | None |
| requirements | | | | |
| attachment | | | | |
| antibody | | | | |
| Target | Humanized | Humanized | Humanized | None |
| capture antibody | modification of | modification | modification of | |
| | Fc to avoid | only | Fc to avoid | |
| | FcγR | | FcγR | |
| Ability to | Yes | No | Yes | No |
| neutralize and | | | | |
| inactivate | | | | |
| microbial target | | | | |
| upon capture by | | | | |
| complement | | | | |
| fixation | | | | |
| Phagocytic | Multiple | Multiple | Multiple | Single |
| compartments | 1. Possibly | 1. Liver (fixed | 1. Possibly | 1. Liver (fixed |
| utilized | circulating | monocytes | circulating | monocyte) |
| | PMN | 2. Spleen (fixed | PMN | 2. Spleen (fixed |
| | 2. Liver (fixed | monocytes) | 2. Liver (fixed | monocytes) |
| | monocytes | | monocytes | |
| | 3. Spleen (fixed | | 3. Spleen (fixed | |
| | monocytes) | | monocytes) | |
| Event triggering | Complement | Transfusion of | Complement | Injection |
| clearance | fixation of | apoptotic cell | fixation of Eg | |
| | target MP RBC | mimic E MP | MP | |
| | complex | ghosts | | |
| Ability to extend | Yes | No | Yes | No |
| passive immunity | | | | |
| period | | | | |
| Process compromises | Not anticipated | Not anticipated | Not anticipated | Yes, RBCs |
| host | | | | loose CR1 |
| immune system | | | | receptor |
| Capability of | Theoretical | Theoretical | Theoretical | Data indicates |
| clearance of | | | | 90% optimized |
| >99.9% of | | | | clearance |
| pathological targets | | | | |
| Host Range | Human and All | Human and All | Human and All | Human and |
| | Animal | Animal | Animal | Animal |
| | | | | Primates Only |

TABLE III
SURFACE RECEPTORS EXPRESSED IN ALL THE PHAGOCYTIC
CELL COMPARTMENTS AND THEIR GRANULAR CONTENT

| | | | | | IC | | | | |
|---|---|---|---|---|---|---|---|---|---|
| | | IC | Chemo- | Chemo- | Adherence | IC | | | |
| | Receptor | Phagocytosis | attractant | attractant | Phagocytosis | Adherence | Enzyme Content of Granules | | |
| | for IgE | Receptor | Receptor | Receptor | Receptor | Phagocytosis | | acid | alkaline |
| | FcgR | FcγR | C3aR | C5aR | CR1 | CR₃ | peroxidase | phosphatase | phosphatase |
| GRANULOCYTE | | | | | | | | | |
| NEUTROPHIL | − | + | + | + | + | + | + | + | + |
| EOSINOPHIL | LOW | + | +? | + | + | + | + | + | |
| | AFFINITY | | | | | | | | |
| BASOPHIL | HIGH | + | + | + | + | + | + | | |
| | AFFINITY | | | | | | | | |
| MAST CELL | HIGH | + | + | + | + | | | + | + |
| | AFFINITY | | | | | | | | |
| MONOCYTE | | | | | | | | | |
| CIRCULATING | + | + | | | + | + | | | |
| BLOOD | | | | | | | | | |
| MONOCYTE | | | | | | | | | |
| KUPFFER | | + | | | + | | | | |
| CELLS IN LIVER | | | | | | | | | |
| INTRAGLOMERULAR | | + | | | + | | | | |
| MESANGIUM | | | | | | | | | |
| OF THE KIDNEY | | | | | | | | | |
| ALVEOLAR | | + | | | + | | | | |
| MACROPHAGES | | | | | | | | | |
| IN THE LUNG | | | | | | | | | |
| SEROSAL | | + | | | + | | | | |
| MACROPHAGES | | | | | | | | | |
| BRAIN | | + | | | + | | | | |
| MICROGLIA | | | | | | | | | |
| SPLEEN SINUS | | + | | | + | | | | |
| MACROPHAGES | | | | | | | | | |
| LYMPH NODE | | + | | | + | | | | |
| SINUS | | | | | | | | | |
| MACROPHAGES | | | | | | | | | |

TABLE IV
SITES FOR POSSIBLE ATTACHMENT OF MP TO THE E SURFACE

17-Beta-Estradiol Receptor
Anion Exchange Protein (AE1)
Aquaporin 1 Channel Protein
Band 3
Blood Group Antigens
Cell Age Specific Surface Protein (part lost in senescent cells)
Ceruloplasm Receptor
Chemokine Receptors
Concanavalin A Receptors
CR1 (Knops System Antigens)
DAF (Cromer System Antigens)
Folate Binding Protein (FBP) Receptors
Glycophorin A Receptor
Hyaluronan Receptor
Integrin Receptor
Interleukin 2 Receptors
Laminin Receptor
Lectin Receptor
Lymphocyte Associated Antigen 3
MIC-2 Protein
MSP-1 Peptide Receptor
Neurothelin
Platelet Glycoprotein IV
Tamm-Horsfall Glycoprotein Receptors
Transferrin Receptor
And Any Other Surface Protein or Carbohydrate

TABLE V
GLOSSARY

| | |
|---|---|
| Ab or a | Any immunoglobulin type (IgG, IgM, IgA, IgE, etc.) or |
| | antibody fragment such as (Fab)₂ or Fab |
| Ag or ag | Any immunogenic molecule with specificity for any |
| | pathologic antibody, often an autoimmune antibody |
| E HP | Sensitization of the erythrocyte with the antibody pair that |
| | binds to the CR1 site on the erythrocyte surface exclusively. |
| E HP (antigen) | Sensitization of the erythrocyte with the antibody and |
| | antigen pair that binds to the CR1 site on the erythrocyte |
| | surface exclusively. |
| E HP Target | Clearance complex to remove the pathologic target from |
| pathologic target | the blood. The target may be microbial or any that is |
| (virus or cell with | immunogenic and determines the specificity of the capture |
| surface antigens) | antibody. |
| E MP (a₁a₂) | Sensitization of the erythrocyte with the antibody pair that |
| | binds to the CR1 site on the erythrocyte surface. |
| E MP (a₁-a₂) Target | Clearance complex to remove the pathologic target from the |
| pathologic target | blood. The target may be microbial or any that is |
| (virus or cell with | immunogenic and determines the specificity of the capture |
| surface antigens) | antibody. |
| E MP (a-ag) | Sensitization of the erythrocyte with the antibody and |
| | antigen pair that binds to any site other than CR1 on the |
| | erythrocyte surface. |
| E MP (a-ag) Target | Clearance complex to remove the pathologic target from the |
| pathologic target | blood. The target is antibody in nature due to the |
| (autoimmune | requirement for binding to the capture antigen and must have |
| antibody specific | specificity for the antigen. |
| for the Ag on the | |
| sensitized E) | |
| E HP (antigen) | Clearance complex to remove the pathologic target from the |
| Target pathologic | blood. The target is antibody in nature due to the |
| target (autoimmune | requirement for binding to the capture antigen and must have |
| antibody specific | specificity for the antigen. |
| for the ag on the | |
| sensitized E) | |
| HP | Heteropolymer/two antibody molecules covalently joined |
| | where one has specificity for CR1 and the other has |
| | specificity for a pathologic target. |
| HP (antigen) | Heteropolymer/one antibody molecules covalently attached |
| | to an antigen where the antibody has CR1 specificity and the |
| | antigen is reactive with some pathologic or autoimmune |
| | antibody. |
| IC | Immune complex, antigen and antibody complex, also |
| | antibody fragment and antigen complex. |
| IC (C3b) | Immune complex where antibody possesses an Fc region and |
| Fc fragment present | fixes and activates complement resulting in deposition of |
| C3b present | C3b. |
| IC (IgG) | Immune complex where antibody possesses an Fc region. |
| Fc fragment present | |
| MP | Molecule pair, comprising of two types MP a₁a₂ and MP a- |
| | ag |
| MP (a₁a₂) | Molecule pair, comprising of two antibodies or antibody |
| | fragments with different specificities covalently attached in |
| | any manner that does not compromise the specific |
| | interaction between the two antibody or fragment |
| | interactions with their immunogenic targets. One antibody |
| | or fragment is specific to a surface protein on the |
| | erythrocyte, excluding the CR1 site, and another antibody or |
| | fragment that is specific to an expressed immunogen on the |
| | surface of the pathologic microbial or other target to be |
| | cleared from the circulating system. |
| MP (a-ag) | Molecule pair, comprising of one antibody and one antigen |
| | where the antibody or fragment is specific to a surface |
| | protein on the erythrocyte, excluding the CR1 site, and it is |
| | covalently coupled to an antigen that is specific to a |
| | pathologic antibody usually autoimmune antibody, without |
| | disruption of either function. |

TABLE VI

| | | | | | | DURATION |
|---|---|---|---|---|---|---|
| | | | | | | OF |
| | | | | | | FUNCTION |
| | IN VIVO | | CLEARANCE | | | OF |
| | CLEARANCE | | MEDIATED | SITE OF | RATE OF | PROTECTIVE |
| | OF | OPSONIZATION | BY | CLEARANCE | CLEARANCE | SYSTEM |
| A | Immune | w/o C3b | Phagocytic | Possibly | Slow | Lifetime |
| | complex | IgG only | cells | PMN in | | |
| | | | | circulation | | |
| | | | | primarily | | |
| | | | | monocyte | | |
| | | | | liver and | | |
| | | | | spleen | | |
| B | Immune | IgG, C3b | Phagocytic | Possibly | More rapid | Lifetime |
| | complex | | cells | PMN in | | |
| | | | | circulation | | |
| | | | | primarily | | |
| | | | | monocyte | | |
| | | | | liver and | | |
| | | | | spleen | | |
| C | Immune | IgG, C3b | E CR1 | Exclusively | Very rapid | Lifetime |
| | complex | | (CR1 | monocytes | | |
| | | | exchange | liver and | | |
| | | | reaction) | spleen | | |
| D | Target | IgG only | E HP | Exclusively | Very rapid | Minutes to |
| | | (C3b not | (CR1 | monocytes | | hours |
| | | required) | exchange | liver and | | |
| | | | reaction) | spleen | | |
| E | Target | IgG, C3b | E MP | Possibly | More rapid | 120 Days |
| | | | Phagocytosis | PMN in | | |
| | | | STE I | circulation | | |
| | | | | primarily | | |
| | | | | monocytes | | |
| | | | | liver and | | |
| | | | | spleen | | |
| F | Target | IgG only, | Eg MP | Liver and | Very rapid | Minutes to |
| | | (C3b not | Phagocytosis | spleen | | hours |
| | | required) | STE IIa | monocytes | | |
| G | Target | IgG, C3b | Eg MP | Liver and | More rapid | Days |
| | | | Phagocytosis | spleen | | (long-lived) |
| | | | STE IIb | monocytes | | |
| H | Target | Complement | Eg MP | Liver and | More rapid | Days |
| | | (Alternate | Phagocytosis | spleen | | (long-lived) |
| | | Pathway) | STE IIb | monocytes | | |

TABLE VII

| Process | | | | | |
|---|---|---|---|---|---|
| Characterization | STE I | STE IIa | STE IIb | STE IIb | HP CR1 |
| Globulin type | Antibody Pair | Antibody Pair | Antibody Pair | Antibody Pair | Antibody Pair |
| (RBC | MP (molecule | MP (molecule | MP (molecule | MP (molecule | HP (hetero- |
| attachment and | pair) | pair) | pair) | pair) | polymer) |
| target capture | | | | | |
| antibodies) | | | | | |
| RBC | All sites other | All sites other | All sites other | All sites other | CR1 only |
| Attachment | than CR1 and | than CR1 and | than CR1 and | than CR1 and | |
| site | artificial site | artificial site | artificial site | artificial site | |
| Attachment | Fab, (Fab)₂, | Fab, (Fab)₂, and | Fab, (Fab)₂, | Fab, (Fab)₂, no | IgG anti CR1 |
| antibody | (devoid of Fc) | IgG with normal | and IgG with | IgG Fc region | only (normal |
| | incapable of | Fc (fix | normal Fc (fix | required | Fc) |
| | fixing | complement) | complement) | | |
| | complement | | | | |
| | | | Fab, (Fab)₂, no | | |
| | | | IgG Fc region | | |
| | | | required | | |
| Pathologic | IgG (must | Fab, (Fab)₂, IgG | Fab, (Fab)₂, | Fab, (Fab)₂, | Fab, (Fab)₂, |
| target capture | possess Fc) or | (normal Fc) | IgG | | IgG |
| antibody | modified Fc | | (normal Fc) | | (normal Fc) |
| | to fix | | | | |
| | complement | | | | |
| Globulin | Injection of | Ex vivo RBC | Ex vivo RBC | Ex vivo RBC | Injection of |
| delivery method | MP or | ghost (high | ghost (low | ghost (low | HP |
| | transfusion of | surface PS) | surface PS) | surface PS) | |
| | E MP | sensitization of | sensitization of | sensitization of | |
| | | universal donor | universal donor | universal donor | |
| | | cells, type O, | cells, type O, | cells, type O, | |
| | | and their | and their | and their | |
| | | transfusion | transfusion | transfusion | |
| Duration of | Long-lasting | Intermediate | Long-lasting | Long-lasting | Short-lived |
| passive | | lasting | | | |
| immunity | | | | | |
| period | | | | | |
| Other antibody | Humanized | Humanized | Humanized | Humanized | None |
| requirements | | | | | |
| attachment | | | | | |
| antibody | | | | | |
| Target | Humanized | Humanized | Humanized | Humanized | None |
| capture | modification of | modification | modification of | antibody | |
| antibody | FC to avoid | only | Fc to avoid | fragment (no Fc | |
| | FcγR | | FcγR | required) | |
| Ability to | Yes | No | Yes | Yes | No |
| neutralize and | | | | | |
| inactivate | | | | | |
| microbial target | | | | | |
| upon capture by | | | | | |
| complement | | | | | |
| fixation | | | | | |
| Phagocytic | Multiple | Multiple | Multiple | Multiple | Single |
| compartments | 1. Possibly | 1. Liver (fixed | 1. Possibly | 1. Possibly | 1. Liver |
| utilized | circulating | monocytes | circulating | circulating | (fixed |
| | PMN | 2. Spleen (fixed | PMN | PMN | monocyte) |
| | 2. Liver (fixed | monocytes) | 2. Liver (fixed | 2. Liver (fixed | 2. Spleen |
| | monocytes | | monocytes | monocytes | (fixed |
| | 3. Spleen (fixed | | 3. Spleen (fixed | 3. Spleen (fixed | monocytes) |
| | monocytes) | | monocytes) | monocytes) | |
| Event triggering | Complement | Transfusion of | Complement | Cessation of | Injection |
| clearance | fixation of | apoptotic cell | fixation of Eg | chloroquine | |
| | target MP RBC | mimic E MP | MP | administration | |
| | complex | ghosts | | | |
| Ability to | Yes | No | Yes | Yes | No |
| extend passive | | | | | |
| immunity | | | | | |
| period | | | | | |
| Process compromises | Not anticipated | Not anticipated | Not anticipated | Not anticipated | Yes, RBCs |
| host | | | | | loose CR1 |
| immune system | | | | | receptor |
| Capability of | Theoretical | Theoretical | Theoretical | Theoretical | Data |
| clearance of | | | | | indicates |
| >99.9% of | | | | | ≧95% |
| pathologic | | | | | clearance |
| targets | | | | | |
| Host Range | Human And | Human And | Human And | Human And | Human And |
| | All Animal | All Animal | All Animal | All Animal | Animal |
| | | | | | Primates |
| | | | | | Only |

What is claimed is:

1. A method for elimination of pathological agents from the blood of a patient comprising administering to said patient at least one sensitized erythrocyte having a molecule pair antibody complex that is capable of binding a pathological agent at a site other than the CR1 receptor, and eliminating said pathological agent from said patient's blood independent of the CR1 exchange reaction.

2. The method of claim 1 including wherein said molecule pair antibody complex comprises two antibodies that are covalently linked, wherein one of said antibodies is specific for binding to an erythrocyte receptor site and the other antibody is specific to said pathological agent.

3. The method of claim 2 including wherein said antibodies are monoclonal antibodies.

4. The method of claim 2 including wherein said patient is a human being or an animal and wherein said antibodies are humanized or non-humanized antibodies.

5. The method of claim 2 including wherein said antibodies may be of any type or an antibody fragment (Fab)₂ or Fab devoid of the Fc region.

6. The method of claim 2 including wherein said erythrocyte receptor site is any immunogenic site on the erythrocyte's surface other than the CR1 receptor.

7. The method of claim 1 including wherein said pathological agent is at least one agent comprising at least one of a microbial organism, a virus, a bacteria, a protein, rickettsia, fungi, parasite; toxin, an agent of biological and chemical warfare, a dysplastic and metastatic cancer cell, an autoimmune antibody, and any molecule capable of mediating a pathologic process or present in the body of said patient.

8. The method of claim 1 including using said molecule pair to sensitize at least one intact erythrocyte or at least one erythrocyte ghost in vivo or ex vivo.

9. The method of claim 1 including wherein said molecule pair is specific for at least one pathological agent.

10. The method of claim 1 including wherein the molecule pair binds to at least one immunogenic epitopes present in erythrocyte surface proteins other than CR1.

11. The method of claim 1 including wherein said sensitized erythrocyte molecule pair is from a human being, a non-human primate or other animal.

12. The method of claim 8 including preparing erythrocyte ghosts having senescence markers and sensitizing said erythrocyte ghosts with said molecule pair.

13. The method of claim 12 including wherein at least one of said erythrocyte is sensitized before or after preparing said erythrocyte ghost.

14. The method of claim 8 including lysing of said erythrocyte in a hypotonic solution to effect the surface appearance of phosphatidylserine and rendering said erythrocyte ghost molecule pair an apoptotic cell mimic.

15. The method of claim 8 including chemically modifying said erythrocyte by addition of phosphatidylserine on the erythrocyte's surface before or after molecule pair sensitization and subsequent erythrocyte lysis.

16. The method of claim 8 including chemically modifying said erythrocyte before or after molecule pair binding by addition of Galactose, α1,3 to effect the creation of a senescence-associated epitope.

17. The method of claim 8 including preparing said erythrocyte ghost in the presence of divalent cation (Mg⁺⁺) and in the absence of ATP resulting in high phosphatidylserine exposure on the erythrocyte ghost molecule pair surface.

18. The method of claim 8 including preparing said erythrocyte ghost in the presence of ATP for providing erythrocyte ghosts having limited surface phosphatidylserine expression.

19. The method of claim 8 including preparing said senescent marker by crosslinking of red blood cell surface protein by hetero-bifunctional cross-linking reagents or antibody cross-linking prior to molecule pair sensitization and subsequent lysis to produce the erythrocyte ghost molecule pair.

20. The method of claim 8 including obtaining at least one apoptotic erythrocyte by density gradient centrifugation and allowing only senescent erythrocytes to be sensitized and subsequently lysed to produce at least one apoptotic mimic erythrocyte ghost molecule pair.

21. The method of claim 8 including providing for the production of at least one apoptotic mimic or natural apoptotic erythrocyte ghost molecule pair.

22. A method for blood-borne pathogen clearance in a patient in vivo comprising:
a) administering to a patient an effective amount of a molecule pair, wherein said molecule pair is prepared using humanized or non-humanized antibodies;
b) allowing said molecule pair to bind to a specific immunogenic site on at least one erythrocyte surface different to CR1 thereby forming a sensitized erythrocyte molecule pair; and
c) allowing said sensitized erythrocyte molecule pair to bind to a specific pathological target in said patient's blood to any site on said erythrocyte other than the CR1 resulting in an erythrocyte-molecule pair-pathological target, and clearing said erythrocyte-molecule pair-pathological target from said patient's blood.

23. A method for blood-borne pathogen clearance in a patient in vivo comprising:
a) sensitizing at least one erythrocyte with a molecule pair ex vivo;
b) administering an effective amount of said sensitized erythrocyte molecule pair to said patient; and
c) allowing said sensitized erythrocyte molecule pair to bind to a specific pathological agent resulting in an erythrocyte-molecule pair-pathological agent, and clearing said erythrocyte-molecule pair-pathological agent from said patient's blood.

24. A method for blood-borne pathogen clearance in a patient in vivo comprising:
a) preparing at least one erythrocyte ghost having senescence markers;
b) sensitizing at least one of said erythrocyte ghosts with at least one molecule pair ex vivo;
c) administering an effective amount of said sensitized erythrocyte ghost molecule pair to a patient; and
d) allowing said sensitized erythrocyte ghost molecule pair to bind to a specific pathological agent present in said patient's blood resulting in an erythrocyte ghost-molecule pair-pathological agent, and clearing said erythrocyte ghost-molecule pair-pathological agent through the privileged apoptotic or senescent cell natural clearance system of said patient's body.

25. The method of claim 8 including wherein said erythrocyte ghost has a surface appearance of phosphatidylserine and wherein the recognition of said phosphatidylserine on the surface of said erythrocyte ghost by macrophage phosphatidylserine receptors is blocked.

26. The method of claim 25 including wherein said molecule pair comprises an Fc region suitable for complement fixation but incapable of being recognized by Fcγ receptors.

27. The method of claim 19 including:
a) wherein said red blood cell surface protein is a band 3 surface protein and wherein said erythrocyte ghost has a surface appearance of phosphatidylserine, and wherein the recognition of said phosphatidylserine on the surface of said erythrocyte ghost by macrophage phosphatidylserine receptors is blocked;
b) administering an effective amount of chloroquine to said patient to temporarily prevent elimination of said pathological agent by said sensitized erythrocyte ghost; and
c) ceasing administration of said chloroquine to said patient, and eliminating said pathological agent from said patient's blood.

28. The method of claim 1 including adding an androgen or androgenic like substance with said molecule pair for increasing the survival time of said erythrocyte molecule pair.

29. A composition comprising an erythrocyte and a molecule pair antibody complex wherein said erythrocyte is capable of being bound to at least one of said molecule pair antibody complex at a site that is other than the CR1 receptor, and wherein said molecule pair antibody complex is capable of binding a pathological agent.

30. The composition of claim 29 wherein said molecule pair antibody complex is comprised of at least one monoclonal antibody.

31. The composition of claim 30 wherein said monoclonal antibodies are humanized or non-humanized antibodies.

32. A composition comprising an erythrocyte ghost and a molecule pair antibody complex wherein said erythrocyte ghost is capable of being bound to at least one of said molecule pair antibody complex at a site that is other than the CR1 receptor, and wherein said molecule pair antibody complex is capable of binding a pathological agent.

33. The composition of claim 32 wherein said erythrocyte ghost has a surface appearance of phosphatidylserine, and wherein said phosphatidylserine on the surface of said erythrocyte ghost is blocked from recognition by macrophage phosphatidylserine receptors.
